(19) United States
(12) Patent Application Publication
(10) Pub. No.: US 2011/0017218 A1
(43) Pub. Date: Jan. 27, 2011

(54) TREATMENT SYSTEM

(75) Inventor: Robert John Oriel Davies, Headington (GB)

Correspondence Address: HARNESS, DICKEY & PIERCE, P.L.C., P.O. BOX 828, BLOOMFIELD HILLS, MI, 48303, US

(73) Assignee: ISIS INNOVATION LIMITED, Oxford (GB)

(21) Appl. No.: 12/866,525

(22) PCT Filed: Feb. 4, 2009
(86) PCT No.: PCT/GB2009/000291

§ 371 Date: Oct. 7, 2010

(30) Foreign Application Priority Data
Feb. 6, 2008 (GB) 0802201.4

(51) Int. Cl. A61M 1/04; A61M 16/00; A61M 16/20

(52) U.S. Cl. 128/207.16; 128/200.24

(57) ABSTRACT

Treatment of a pneuomothroax or other condition wherein unwanted fluid is present in a pleural cavity is achieved using a system comprising a chest device (1) and a mouth device (3). The chest device comprises a catheter having at least one opening and a one-way outlet valve arrangement. The mouth device comprises a body defining a cavity, a mouthpiece opening into the cavity, a one-way inlet valve arrangement, and a one-way outlet valve arrangement opening at a positive pressure greater than the outlet valve arrangement of the chest device. The catheter of the chest device is inserted through the chest to open into the pleural cavity (50). The mouthpiece of the mouth device is placed in the mouth. The patient inhales and exhales through the mouth device. During exhalation a positive pressure is developed in the chest against the outlet valve arrangement of the mouth device, which drives the unwanted fluid out of the pleural cavity through the chest device.

The present invention relates to the treatment of conditions in a patient in which unwanted fluid is present in a pleural cavity between a parietal pleura and a visceral pleura. Such conditions include a pneumothorax in which the unwanted fluid is air, and pleural effusion in which the unwanted fluid could be a liquid such as pleural fluid induced by infected pleural fluid, pus or blood.

Pneumothorax is an abnormal collection of air between the lung and the wall of the chest, in particular in one of the pleural cavities between a parietal pleura and a visceral pleura (or occasionally both pleural cavities). Normally the pleural cavity is substantially empty, containing only a few mL of pleural fluid. However, it is a “potential” space into which fluid such as air can be introduced, most commonly by perforation of the outer surface of the lung and the visceral pleura, or by perforation of the chest wall and the parietal pleura by medical procedures or trauma. When air is so introduced, the negative pressure in the pleural cavity acts to draw air into the pleural cavity which results in the lung becoming separated from the chest wall and deflating. This impairs its function and can cause breathlessness, which can sometimes be serious or even fatal.

Pneumothorax develops in a number of different ways. The most common cause of pneumothorax is perforation of the lung and the visceral pleura by a medical procedure such as by a needle inserted into the chest for the medical diagnosis or treatment of a range of diseases (iatrogenic pneumothorax). Pneumothorax also occurs in apparently healthy people with mild structural abnormalities of the lung (primary spontaneous pneumothorax), or due to significant underlying lung disease resulting in an air leak across the visceral pleura from the surface of the lung (secondary spontaneous pneumothorax), or following or due to chest wall injury (traumatic pneumothorax). The development of pneumothorax via one of these mechanisms is common. It is estimated that there are about 24 spontaneous pneumothoraces per 100,000 of the population in the Western world, and iatrogenic pneumothorax is more common than spontaneous pneumothorax, although its exact incidence is not known. Therefore, each year in the US and the UK alone there are more than 170,000 pneumothoraces for which treatment is needed.

The development of a pneumothorax impairs function of the underlying lung and normal respiratory movement of the wall of the chest and the diaphragm. In advanced cases impairment of circulatory blood flow through the heart can also be impaired. These problems induce impaired respiratory function, which leads to breathlessness and impaired gas exchange and which can be severe, and in the case of impaired circulatory function, hypotension. These effects are occasionally fatal.

The standard treatment of pneumothorax consists of the removal of the abnormal air in the pleural cavity either by manual aspiration, that is manual suction of air from the pleural cavity, or by chest tube drainage, that is the insertion of a tube into the pleural cavity down which air can spontaneously pass. The usual initial treatment for patients who are not severely ill is manual aspiration.

The standard method of manual aspiration involves insertion of a catheter through the chest of the patient and through the parietal pleura so that the catheter opens into the pleural cavity. Then a negative pressure is generated in the pleural cavity by applying suction, often with a plastic syringe attached via a three-way tap and plastic tubing to an improvised one-way valve consisting of sterile water/saline through which aspirated air is bubbled. This is capable in many instances of removing the unwanted air from the pleural cavity, but suffers from problems due to negative pressure in the pleural cavity generating an increased pressure gradient across the visceral pleura of the lung, i.e. between the lung cavity and the pleural cavity. This pressure gradient tends to exacerbate the original air leak as the increased pressure gradient across the visceral pleura encourages air to replenish the pneumothorax as it is aspirated, for example through the puncture in the visceral pleura through which the air was originally introduced, or through a further puncture in the visceral pleura induced by the negative pressure in the pleural cavity generated by the aspiration itself. Such manual aspiration is also time-consuming, typically requiring something of the order of 20-45 minutes of medical staff time to perform the aspiration.

In other conditions, other fluids such as infected pleural fluid, pus or blood may need to be removed from the pleural cavity. In general terms, such conditions are treated in a similar manner to a pneumothorax, most often by aspiration of the fluid or by chest tube drainage.

According to the present invention, there is provided a system for treating a condition in a patient in which unwanted fluid is present in a pleural cavity between a parietal pleura and a visceral pleura, the system comprising a chest device and a mouth device,

the chest device comprising: a catheter defining a lumen and having at least one opening into the lumen; and an outlet valve arrangement allowing one-way flow of fluid out of the lumen and opening at a first positive pressure, and

the mouth device comprising: a body defining a cavity; a mouthpiece opening into the cavity; an inlet valve arrangement allowing one-way flow into the cavity; and an outlet valve arrangement allowing one-way flow out of the cavity and opening at a second positive pressure greater than the first positive pressure at which the outlet valve arrangement of the chest device opens.

The system may be used in a method to treat the condition, in accordance with a further aspect of the invention. In the method: the catheter of the chest device is inserted through the chest of the patient and through the parietal pleura so that the at least one opening opens into the pleural cavity, and placing the mouthpiece of the mouth device in the mouth of the patient; and the patient inhales and exhales through the mouth device. This has the result that during exhalation a positive pressure is developed in the chest cavity, consisting of the lung cavity and pleural cavity, against the outlet valve arrangement of the mouth device. This positive pressure drives the unwanted fluid out of the pleural cavity through the chest device. During inhalation the positive pressure in the lungs ceases and a negative pressure is developed in the lungs. As a result, the outlet valve of the chest device closes so air is prevented from being re-introduced into the pleural cavity through the chest device.

Effectively, therefore, the unwanted fluid is expelled from the pleural cavity by the patient's own effort when exhaling. This contrasts with the known manual aspiration technique for treating a pneumothorax described above in which the effort is applied by suction through the catheter. This is achieved due to the novel system comprising a combination of the chest device and the mouth device in which the outlet valve arrangement of the mouth device opening at the second positive pressure which is greater than the first positive pressure at which the outlet valve arrangement of the chest device opens, so that the pressure in the lung and the pleural cavity rises above the level needed to open the outlet valve arrangement of the chest device.

The use of the system prevents the generation of an increased pressure gradient across the visceral pleura during fluid removal. This reduces the likelihood of further air replenishing the pneumothorax as it is aspirated, as compared to the known manual aspiration technique for treating a pneumothorax described above.

The use of the system has a number of advantages. The system allows the removal of the unwanted fluid by the patient's own respiratory movement, merely after insertion of the chest device. Only brief patient training is required. This significantly reduces the burden on medical staff; for example as compared to the manual aspiration technique described above. The work required is simpler and may be performed by less qualified staff, requiring supervision by, for example, nursing staff instead of performance of manual aspiration by a doctor. This improves the cost effectiveness of the procedure by minimising the use of medical staff time. The advantages of this can be considerable when, for example, a pneumothorax develops during a medical procedures list which is then interrupted for a sustained period. This invention allows medical staff to continue with the list while the pneumothorax is treated.

The system may be used for treatment of a pneumothorax in which case the unwanted fluid is air. However the system is of general application to any unwanted fluid in a pleural cavity, including liquids such as pleural fluid generated as a result of cancer, infected pleural fluid, pus or blood, and including mixtures of liquid and gas. For example, the system may have a useful clinical niche in initiating drainage from permanently inserted chest catheters used to manage cancer related fluid by patients themselves in their own home, in which situation patients often have practical difficulties in applying suction to the catheter.

To allow better understanding, an embodiment of the present invention will now be described by way of non-limitative example with reference to the accompanying drawings, in which:

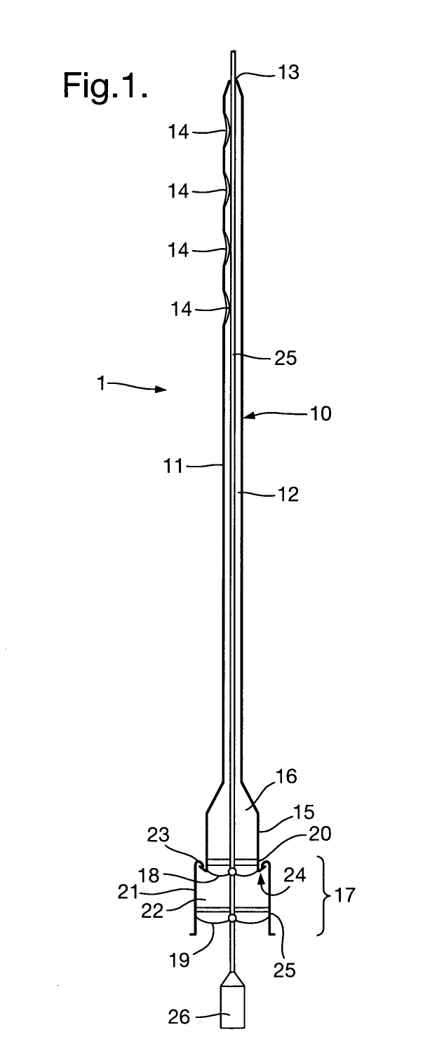

FIG. 1 is a cross-sectional view of a chest device;

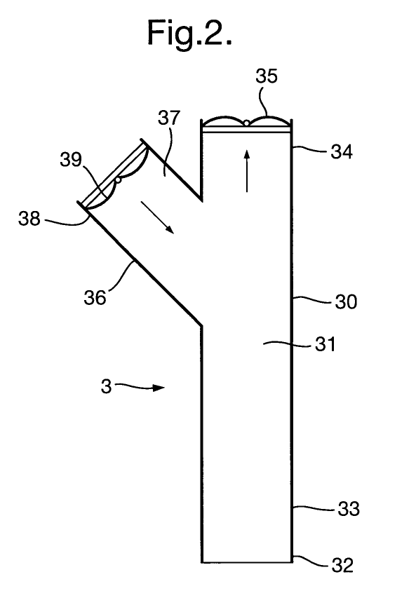

FIG. 2 is a cross-sectional view of a mouth device;

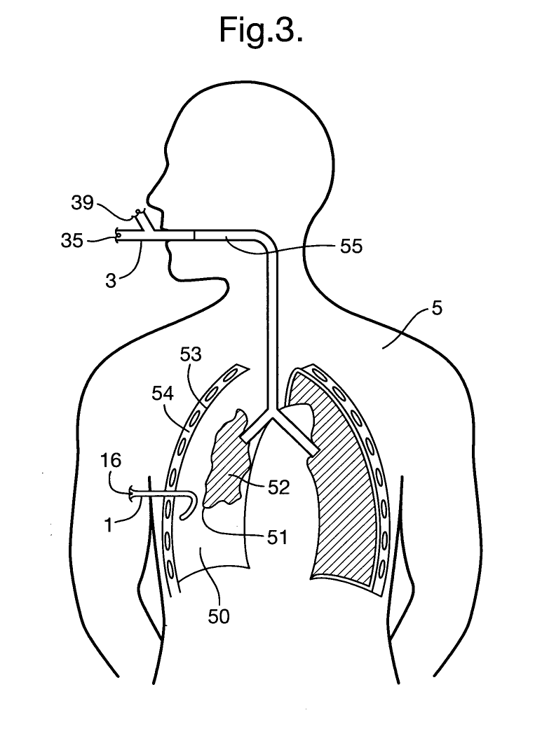

FIG. 3 is a cross-sectional view of the chest device inserted into a pleural cavity; and

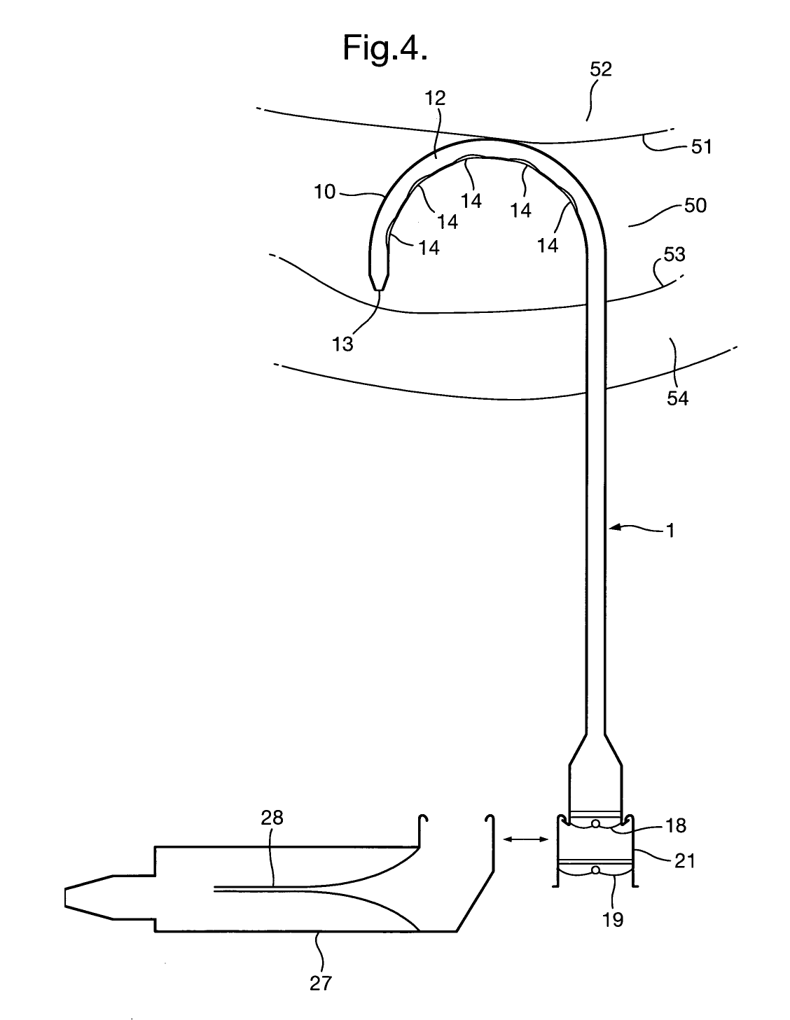

FIG. 4 is a schematic cross-sectional view of a patient being treated using the chest device and mouth device.

The system consists of a chest device 1 as shown in FIG. 1 and a mouth device 3 as shown in FIG. 2

The chest device 1 is constructed as follows. The chest device 1 comprises a catheter 10 formed by a cylindrical wall 11 defining a central lumen 12. The catheter 10 has a terminal opening 13 at a distal end and lateral openings 14 in the wall 11 adjacent the distal end, opening into the lumen 12. The catheter 10 is of sufficiently small diameter to be inserted through a chest wall and parietal pleura. To achieve this, the outer diameter of the catheter 10 is at most 12 mm, preferably at most 5 mm in practical embodiments. The catheter 10 is sufficiently flexible to be curled into a curved shape after insertion into a pleural cavity, without blocking the lumen 11.

The chest device further comprises a body 15 connected to the proximal end of the catheter 10, the body 15 defining a cavity 16 communicating with the lumen 12 of the catheter 10. The body 15 supports an outlet valve arrangement 17 consisting of an inner valve 18 and an outer valve 19, each being a diaphragm valve consisting of a respective diaphragm which allows one-way flow of fluid out of the lumen 12 in the catheter 10.

The inner valve 18 is disposed across and closes an open end 20 of the cavity 16 in the body 15.

The outer valve 19 is supported on a tubular support 21 defining a cavity 22. One end 23 of the tubular support 21 is connected around the open end 20 of the cavity 16 in the body 15 by a clip 24 so that the cavity 16 in the body 15 communicates with the cavity 22 in the tubular support 21 through the inner valve 18. The outer valve 19 is disposed across and closes the other end 25 of the cavity 22 in the tubular support 21. In this manner, the inner valve 18 and outer valve 19 are arranged in series to fluid flow out of the lumen 12.

The clip 24 is detachable to allow the tubular support 21 to be removed from the body 15 so that only the inner valve 18 of the outlet valve arrangement remains on the chest device 1.

To assist insertion into a pleural cavity, the chest device 1 is supplied loaded on a needle 25 extending inside the bore 12 of the catheter 10, along the entire length of the catheter 10 and the chest device 1. The distal end of the needle 25 protrudes from the terminal opening 13. The proximal end of the needle 25 protrudes beyond the body 15 and outlet valve arrangement 16 where it is connected to a handle 26 of larger cross-section than the needle 25 to allow manipulation and connection of a syringe. The needle 25 is a safety needle, for example of the Verres type, to allow introduction into the pleural cavity with minimum risk.

The mouth device 3 is constructed as follows. The mouth device 3 comprises a body 30 made of moulded plastic and is hollow, defining a cavity 31. The body 30 is generally tubular.

The body 30 has at its proximal end 33 a mouthpiece 32 formed simply by leaving the proximal end 33 open, although the mouthpiece 32 could alternatively be shaped as known for mouthpieces for other uses.

The body 30 is closed at its distal end 34 by an outlet valve 35 being a diaphragm valve consisting of a diaphragm which allows one-way flow of fluid out of the cavity 31 in the body 30.

On one side thereof, the body 30 has a leg 36 branching away from the remainder of the body 30. The leg 36 is hollow, defining a space 37 which is part of the cavity 31. The leg 36 is closed at its outer end 38 by an inlet valve 39 being a diaphragm valve consisting of a diaphragm which allows one-way flow of fluid into the cavity 31 in the body 30.

The chest device 1 and mouth device 3 are supplied in combination, typically being packaged together in a sterile package.

The method of using the system will now be described.

As shown schematically in FIG. 3, the system is applied to a patient 5 having a condition in which a pleural cavity 50 contains a fluid, for example air in the case of a pneumothorax. The pleural cavity 50 is defined between the visceral pleura 51 which surrounds the lung 52 and the parietal pleura 53 inside the chest wall 54. In particular the chest device 1 is inserted into the pleural cavity 50 and the mouth device is placed into the mouth 55 of the patient 5 as will now be described.

The chest device 1 is inserted into the pleural cavity 50 of the patient 5 by inserting the needle 25, having the chest device 1 loaded thereon, through the chest wall 54 and the parietal pleura 53 in the second intercostal space. The needle 25 pierces the tissue of the chest wall 54 and the parietal pleura 53 and carries the distal end of the catheter 10 inside the pleural cavity 50. Thereafter, the needle 25 is slidable withdrawn from the chest device 1 which is left inside the pleural cavity 50, as shown in FIG. 4. The openings 13 and 14 open into the pleural cavity 50 so fluid in the pleural cavity 50 can flow through the openings 13 and 14 into the lumen 12.

Due to its flexibility, the catheter 10 curls into a curved shape after insertion. In this curved configuration, the openings 13 and 14 have a reduced risk of becoming blocked by the visceral pleura 51 or the parietal pleura 53 coming together when the fluid is removed from the pleural cavity 50. Thus the chest device overcomes some of the problems with some existing manual aspiration devices that they are cumbersome, with the catheter kinking at the time of the procedure, and that it can fail to completely remove the air in the pleural cavity if the gradually expanding lung impacts on the open end of the chest wall catheter.

The mouth device 3 is applied simply by placing the mouthpiece 32 thereof into the mouth 55 of the patient 5.

The system is used simply by the patient inhaling and exhaling through the mouth device 1. This drives fluid out of the pleural cavity as a result of the positive pressures at which various valves of the chest device 1 and mouth device 3 open, as will now be described.

The outlet valve arrangement 16 of the chest device 1 as a whole opens at a first positive pressure. The outlet valve 35 of the mouth device 3 opens at a second positive pressure which is greater than the first positive pressure. This has the result that during exhalation a positive pressure is developed in the chest cavity consisting of the lungs 52 and the pleural cavity 50, with no pressure gradient across the visceral pleura 51, against the outlet valve 35 of the mouth device 3. The positive pressure so generated in the pleural cavity 50 is greater than the first positive pressure at which the outlet valve arrangement 16 of the chest device 1 opens and hence drives the unwanted fluid out of the pleural cavity 50 through the chest device 1. This expands the collapsed lung 52 into the pleural cavity 50 in a gentle manner with minimum damage to tissues.

The inlet valve 39 of the mouth device 3 opens at a third positive pressure, allowing the patient to inhale. During inhalation the positive pressure in the chest ceases and a negative pressure is developed in the chest. As a result, the outlet valve arrangement 16 of the chest device 1 closes. This prevents air from being re-introduced into the pleural cavity 50 through the chest device 1.

Thus, fluid is driven out of the pleural cavity 50 by the effort of the patient 5 breathing. Furthermore this removal of fluid occurs without a negative pressure being generated in the pleural cavity 50. In the event of a pneumothorax caused by a puncture in the visceral pleura 51 or in the event of a puncture induced by negative pressure in the pleural cavity 50 during aspiration, such negative pressure would create a negative pressure gradient across the puncture, tending to urge more fluid back into the pleural cavity 50, thereby acting against the treatment.

Thus the treatment removes fluid from the pleural cavity 50 relatively quickly and with minimal medical supervision beyond training of the patient 5 to breath through the mouth device 3, for example as compared to the known treatment of a pneumothorax by manual aspiration.

The values of the first to third positive pressures may be controlled by suitable design of the outlet valve arrangement 16 of the chest device 1, and the outlet valve 35 and inlet valve 36 of the mouth device 3. In general, the first and second positive pressures may have any value in which the second positive pressure is greater than the first positive pressure and the third positive pressure may have any value allowing the patient to inhale.

To achieve a positive action, the first positive pressure at which the outlet valve arrangement 16 of the chest device 1 opens is typically at least 0.5 cmH₂O, preferably at least 1 cmH₂O. This contrasts with a simple one-way valve, such as a flutter valve, which would open at a lower positive pressure, in fact nearly 0 cmH₂O as compared to the pressure developed in the lung in normal breathing. However, the chest device 1 would still operate correctly if the first positive pressure was less than 0.5 cmH₂O, for example if the outlet valve arrangement 16 shown in FIG. 1 was replaced by a simple one-way valve.

The second positive pressure is typically at least 2 cmH₂O, preferably at least 4 cmH₂O to achieve a margin over the first positive pressure. The second positive pressure could be higher, and could be anything up to maximal volitional expiratory pressure (of the order of 150 cmH₂O), but to minimise any difficulty in the breathing of the patient 5 is typically at most 75 cmH₂O.

The third positive pressure is typically low, for example less than 0.5 cmH₂O, preferably nearly 0 cmH₂O.

The outlet valve arrangement 16 of the chest device 1 will now be considered further. In the outlet valve arrangement 16, the outer valve 19 opens at the second positive pressure and the inner valve 18 opens at a fourth positive pressure less than the second positive pressure. As a result the outlet valve arrangement 16 opens at the second positive pressure and the inner valve 18 has substantially no effect whilst the outer valve 19 is present. However the reason for this construction of the outlet valve arrangement 16 is that the outer valve 19 may be detached by detaching the tubular support 21 from the clip 24. Thereafter, the chest device 1 is left with only the inner valve 18 in place. The fourth positive pressure is chosen to be sufficiently low to allow the chest device 1 to operate in the same manner as a conventional chest drainage device. Thus the fourth positive pressure is less than 0.5 cmH₂O, preferably nearly 0 cmH₂O.

There are several situations where this arrangement allowing conversion of the chest device 1 is useful. Firstly, even after successful treatment, it may be useful to maintain the chest device 1 in place for a period afterwards, for example to complete the treatment or as a precautionary measure. Secondly, depending on the severity of the condition, in some cases aspiration of air from the pleural cavity 50 by this, or any other method, will prove unsuccessful due to a persisting leak of air from the lung 52. In this situation, the standard treatment is the insertion of a chest tube which is then attached to an underwater seal to allow air to be removed from the chest until the puncture in the lung spontaneously heals. This may require admission to hospital with commensurate expense and patient inconvenience. In this situation, the detachable outer valve 19 allows the chest device 1 to be converted to an ambulatory one-way chest wall valve, allowing discharge from hospital and a domiciliary ambulatory pneumothorax treatment in many cases. This can avoid some hospital admissions.

Optionally, the tubular support 21 and outer valve 19 may be replaced by a unit incorporating another valve, for example a unit 27 as shown in FIG. 4 which includes a low-pressure flutter valve 28 and which clips by clip 24 onto the body 15 to dispose the flutter valve 28 across the chest device 1.

Of course the chest device 1 and mouth device 3 may be modified in a variety of ways. Some non-limitative examples of such modifications are as follows.

The various valves 18, 19, 35 and 39 are all described and illustrated as diaphragm valves, but equally be replaced by any type of valve capable of opening at the desired pressure, including without limitation a flutter valve, a reed valve, a spring valve, or a ball valve.

The mouth device 3 may be modified to include an oxygen port on the mouthpiece 32 to allow connection of a supply of oxygen, for example for a breathless patient who requires supplemental oxygen during breathing.

Whilst the invention is described above as relating to treatment of a condition in a patient in which unwanted fluid is present in a pleural cavity, the invention relates equally to treatment of a condition in a patient in which unwanted fluid is present in any cavity in the patient, including without limitation the peritoneum, bladder, bowel and paranasal sinuses. In this case, there is used the same system including a mouth device as described above and a device for insertion into the cavity which is essentially the same as the chest device except perhaps for modifications to the catheter (eg in length, diameter and/or flexibility) to adapt it for insertion into the cavity concerned. The method is the same except of course that the catheter is inserted into the cavity concerned.

1. A method of treating a pneumothorax in a patient in which unwanted air is present in a pleural cavity between a parietal pleura and a visceral pleura, the method comprising:
providing a chest device and a mouth device,
the chest device comprising: a catheter having an outer diameter of 12 mm or less, the catheter defining a lumen and having at least one opening into the lumen, the catheter being adapted to be bent into a curved shape after insertion into the pleural cavity; and an outlet valve arrangement allowing one-way flow of air out of the lumen and opening at a first positive pressure, and
the mouth device comprising: a body defining a cavity; a mouthpiece opening into the cavity; an inlet valve arrangement allowing one-way flow into the cavity; and an outlet valve arrangement allowing one-way flow out of the cavity and opening at a second positive pressure greater than the first positive pressure at which the outlet valve arrangement of the chest device opens;
inserting the catheter of the chest device through the chest of the patient and through the parietal pleura so that the at least one opening opens into the pleural cavity, and placing the mouthpiece of the mouth device in the mouth of the patient; and
the patient inhaling and exhaling through the mouth device, whereby during exhalation a positive pressure is developed in the lungs and the pleural cavity against the outlet valve arrangement of the mouth device which drives the unwanted air out of the pleural cavity through the chest device.

2. A method according to claim 1, wherein the outlet valve arrangement of the mouth device opens at a second positive pressure of 2 cmH₂O or higher.

3. (canceled)

4. A method according to claim 1, wherein the outlet valve arrangement of the mouth device opens at a second positive pressure of 150 cmH₂O or lower.

5. A method according to claim 1, wherein any one or more of the outlet valve arrangement of the mouth device the inlet valve arrangement of the mouth device, and the outlet valve arrangement of the chest device comprises a diaphragm valve.

6. A method according to claim 1, wherein the inlet valve arrangement of the mouth device opens at a third positive pressure of less than 0.5 cmH₂O.

7. (canceled)

8. A method according to claim 1, wherein the outlet valve arrangement of the chest device opens at a first positive pressure of 0.5 cmH₂O or higher.

9. (canceled)

10. (canceled)

11. A method according to claim 1, wherein the outlet valve arrangement of the chest device comprises two valves arranged in series, the outer valve opening at the first positive pressure and the inner valve opening at a fourth positive pressure less than the first positive pressure, the outer valve being detachable leaving the inner valve in place.

12. A method according to claim 11, wherein the inner valve of the outlet valve arrangement of the chest device opens at a fourth positive pressure of less than 0.5 cmH₂O.

13. (canceled)

14. A method according to claim 1, wherein the catheter has an outer diameter of 5 mm or less.

15. A method according to claim 1, wherein the at least one opening of the catheter includes one or more openings in the wall of the catheter.

16. (canceled)

17. (canceled)

18. A system for treating a pneumothorax in a patient in which unwanted air is present in a pleural cavity between a parietal pleura and a visceral pleura, the system comprising a chest device and a mouth device,
the chest device comprising: a catheter having an outer diameter of 12 mm or less, the catheter defining a lumen and having at least one opening into the lumen the catheter being adapted to be bent into a curved shape after insertion into the pleural cavity; and an outlet valve arrangement allowing one-way flow of air out of the lumen and opening at a first positive pressure, and
the mouth device comprising: a body defining a cavity; a mouthpiece opening into the cavity; an inlet valve arrangement allowing one-way flow into the cavity; and an outlet valve arrangement allowing one-way flow out of the cavity and opening at a second positive pressure greater than the first positive pressure at which the outlet valve arrangement of the chest device opens.

19. A system according to claim 18, wherein the outlet valve arrangement of the mouth device opens at a second positive pressure of 2 cmH₂O or higher.

20. (canceled)

21. A system according to claim 18, wherein the outlet valve arrangement of the mouth device opens at a second positive pressure of 150 cmH₂O or lower.

22. A system according to claim 18, wherein any one or more of the outlet valve arrangement of the mouth device, the inlet valve arrangement of the mouth device, and the outlet valve arrangement of the chest device comprises a diaphragm valve.

23. A system according to claim 18, wherein the inlet valve arrangement of the mouth device opens at a third positive pressure of less than 0.5 cmH₂O.

24. (canceled)

25. A system according to claim 18, wherein the outlet valve arrangement of the chest device opens at a first positive pressure of 0.5 cmH₂O or higher.

26. (canceled)

27. (canceled)

28. A system according to claim 18, wherein the outlet valve arrangement of the chest device comprises two valves arranged in series, the outer valve opening at the first positive pressure and the inner valve opening at a fourth positive pressure less than the first positive pressure, the outer valve being detachable leaving the inner valve in place.

29. A system according to claim 28, wherein the inner valve of the outlet valve arrangement of the chest device opens at a fourth positive pressure of less than 0.5 cmH₂O.

30. (canceled)

31. A system according to claim 18, wherein the catheter has an outer diameter of 5 mm or less.

32. A system according to claim 18, wherein the at least one opening of the catheter includes one or more openings in the wall of the catheter.

33. (canceled)

34. A system according to claim 18, wherein the chest device and the mouth device are packaged together.

35. (canceled)

36. (canceled)

37. A mouth device for treating a pneumothorax in a patient in which unwanted air is present in a pleural cavity between a parietal pleura and a visceral pleura the mouth device comprising: a body defining a cavity; a mouthpiece opening into the cavity; an inlet valve arrangement allowing one-way flow into the cavity; and an outlet valve arrangement allowing one-way flow out of the cavity and opening at a second positive pressure greater than the first positive pressure at which the outlet valve arrangement of the chest device opens.

38. A mouth device according to claim 37, wherein the outlet valve arrangement of the mouth device opens at a second positive pressure of 2 cmH₂O or higher.

39. (canceled)

40. A mouth device according to claim 37, wherein the outlet valve arrangement of the mouth device opens at a second positive pressure of 150 cmH₂O or lower.

41. A mouth device according to claim 37, wherein either one or both of the outlet valve arrangement of the mouth device and the inlet valve arrangement of the mouth device comprises a diaphragm valve.

42. A mouth device according to claim 37, wherein the inlet valve arrangement of the mouth device opens at a third positive pressure of less than 0.5 cmH₂O.

43. (canceled)

44. (canceled)

45. A chest device for treating a pneumothorax in a patient in which unwanted #1-61-4 air is present in a pleural cavity between a parietal pleura and a visceral pleura, the chest device comprising: a catheter having an outer diameter of 12 mm or less, the catheter defining a lumen and having at least one opening into the lumen, the catheter being adapted to be bent into a curved shape after insertion into the pleural cavity; and an outlet valve arrangement allowing one-way flow of air out of the lumen and opening at a first positive pressure.

46. A chest device according to claim 45, wherein the outlet valve arrangement of the chest device opens at a first positive pressure of 0.5 cmH₂O or higher.

47. (canceled)

48. A chest device according to claim 45, wherein the outlet valve arrangement comprises a diaphragm valve opening at the first positive pressure.

49. A chest device according to claim 45, wherein the outlet valve arrangement of the chest device comprises two valves arranged in series, the outer valve opening at the first positive pressure and the inner valve opening at a fourth positive pressure less than the first positive pressure, the outer valve being detachable leaving the inner valve in place.

50. A chest device according to claim 49, wherein the inner valve of the outlet valve arrangement of the chest device opens at a fourth positive pressure of less than 0.5 cmH₂O.

51. (canceled)

52. A chest device according to claim 45, wherein the catheter has an outer diameter of 5 mm or less.

53. A chest device according to claim 45, wherein the at least one opening of the catheter includes one or more openings in the wall of the catheter.

54. (canceled)

55. (canceled)
